(19) United States
(12) Patent Application Publication
(10) Pub. No.: US 2023/0183799 A1
(43) Pub. Date: Jun. 15, 2023

(54) PARALLEL SAMPLE AND INDEX SEQUENCING

(71) Applicant: Illumina, Inc., San Diego, CA (US)

(72) Inventors: Shaun E. Hunter, San Diego, CA (US); Paul Sangiorgio, San Diego, CA (US); Aathavan Karunakaran, Berkeley, CA (US); David Bracher, San Diego, CA (US)

(21) Appl. No.: 18/063,581

(22) Filed: Dec. 8, 2022

Related U.S. Application Data
(60) Provisional application No. 63/288,213, filed on Dec. 10, 2021.

(51) Int. Cl. C12Q 1/6874

(52) U.S. Cl. CPC C12Q 1/6874; C12Q 1/6806

(57) ABSTRACT

Systems and methods of identifying nucleobases in a template polynucleotide are disclosed. In one embodiment, such a method may include providing a substrate comprising a plurality of the template polynucleotides in a cluster. The method may further include generating light to stimulate fluorescent emissions from the cluster. The method may further include receiving a first signal emitted at a first intensity from a first plurality of nucleotide analogs hybridized to the plurality of template polynucleotides at a first site. The method may further include receiving a second signal emitted at a second intensity from a second plurality of nucleotide analogs hybridized to the plurality of template polynucleotides at a second site. The method may further include identifying the nucleobases hybridized at the first and second sites of the template polynucleotide based on a combination of the first and second signals.

### CROSS-REFERENCE TO RELATED APPLICATIONS

This application claims priority to U.S. Provisional Application No. 63/288,213, filed Dec. 10, 2021, the content of which is incorporated by reference in its entirety.

### BACKGROUND

### Field

The disclosed technology relates to the field of nucleic acid sequencing. More particularly, the disclosed technology relates to using next generation sequencing to determine the nucleotide sequences of an index sequence and a corresponding target nucleic acid in the same sequencing run.

### Description of the Related Art

In some types of next-generation sequencing technologies, a DNA cluster may be created on a flow cell following amplification of a target polynucleotide. The next generation sequencing cycles may be performed as complementary strands of the target polynucleotides are being synthesized. In each sequencing cycle, deoxyribonucleic acid analogs conjugated with fluorescent labels may be hybridized to the target polynucleotides at a certain site, and excitation light sources may be used to excite the fluorescent labels on the deoxyribonucleic acid analogs. Detectors may capture fluorescent emissions from the fluorescent labels in order to identify the deoxyribonucleic acid analogs. As a result, the sequence of the target polynucleotide may be determined by repeatedly performing sequencing cycles. The speed of sequencing the target polynucleotide may be limited due to the need for performing sequencing cycles and identifying the nucleobases of the target polynucleotide in a serial order.

### SUMMARY

The disclosed technology relates to systems and methods for efficiently determining the nucleobase sequence of a polynucleotide. More particularly, the disclosed technology relates to systems and methods for determining the nucleobase sequences of different portions of a polynucleotide, such as an index portion a template portion, in parallel.

In one aspect, the disclosed technology provides systems and methods of identifying nucleobases in a template polynucleotide. In one embodiment, the disclosed method can include providing a substrate comprising a plurality of the template polynucleotides in a cluster. The method can further include generating light to stimulate fluorescent emissions from the cluster. The method can further include receiving a first signal emitted at a first intensity from a first plurality of nucleotide analogs hybridized to the plurality of template polynucleotides at a first site. The method can further include receiving a second signal emitted at a second intensity from a second plurality of nucleotide analogs hybridized to the plurality of template polynucleotides at a second site. The method can further include identifying the nucleobases hybridized at the first and second sites of the template polynucleotide based on a combination of the first and second signals.

In another aspect, the disclosed technology provides systems and methods of determining the sequence of a template polynucleotide comprising a barcode index. In one embodiment, the disclosed method can include hybridizing an index primer and a read primer to the template polynucleotide. The method can further include extending the index primer with a first labeled nucleotide comprising a first label. The method can further include extending the read primer with a second labeled nucleotide comprising a second label. The method can further include generating light to stimulate fluorescent emissions from the first and second labeled nucleotides. The method can further include determining the sequence of nucleotides in the barcode index and the template polynucleotide by capturing the fluorescent emissions.

The systems, devices, kits, and methods disclosed herein each have several aspects, no single one of which is solely responsible for their desirable attributes. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

It is to be understood that any features of the systems disclosed herein may be combined together in any desirable manner and/or configuration. Further, it is to be understood that any features of the methods disclosed herein may be combined together in any desirable manner. Moreover, it is to be understood that any combination of features of the methods and/or the systems may be used together, and/or may be combined with any of the examples disclosed herein. It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 shows a block diagram which schematically illustrates an example sequencing system that may be used to perform the disclosed methods.

FIG. 2 shows a block diagram which schematically illustrates an example imaging system that may be used in conjunction with the example sequencing system of FIG. 1.

FIG. 3 shows a functional block diagram of an example computer system that may be used in the example sequencing system of FIG. 1.

FIG. 4 shows a schematic of a nucleic acid cluster of an indexed sample and primers used to sequence the indexed sample according to one embodiment of the disclosed technology.

FIG. 5 shows an example dye labeling scheme that may be used in conjunction with the embodiment of FIG. 4.

FIG. 6 is a plot showing graphical representations of sixteen distributions of signals from a nucleic acid cluster according to one embodiment of the disclosed technology.

FIG. 7 is a flow diagram showing a method for sequencing a polynucleotide according to one embodiment of the disclosed technology.

### DETAILED DESCRIPTION

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

### Introduction

In some next-generation sequencing technologies, the addition of a unique identifier, or index sequence, to DNA fragments generated during library preparation can form indexed template sequences which allow multiple DNA libraries to be pooled and sequenced together. In some systems, each template sequence has a unique index portion. The template may be bound to a flow cell and a cluster of identical indexed template sequences generated at a single location on the flow cell. Thus, a DNA cluster on the flow cell may include identical copies of a template polynucleotide having an index sequence portion and an insert portion derived from the original DNA fragments taken from biological or other samples. Further details regarding indexed sequencing of nucleic acids can be found in U.S. Pat. Number 8,822,150, which is incorporated herein by reference.

In one aspect, the disclosed technology can reduce the time for sequencing a template polynucleotide by allowing for simultaneous sequencing of the index sequence portion and the insert portion of the template polynucleotide. In one aspect, the primer for sequencing the index sequence portion (i.e., the index primer) and the primer for sequencing the insert portion (i.e., the read primer) are annealed to the template polynucleotide in the same reaction step to increase the efficiency of sequencing-by-synthesis (SBS) workflows. In addition, annealing the index primer and the read primer to the template strand at the same time may reduce the fluidic complexity of the overall system. For example, the index primer and the read primer may be hybridized to the template polynucleotide simultaneously, and then the index sequence portion and the insert portion may be read-out through SBS chemistry cycles. In one embodiment, in order to separate the signals received from the labeled nucleobases on the index sequence portion and the insert portion, the signal from the index sequence portion is diminished, e.g., by 50% in comparison to the signal generated by the read portion. For example, the index primer may be prepared such that a fraction (e.g., 50%) of the index primer molecules are blocked and cannot be extended. By blocking the addition of labeled nucleobases to the index primer (or read primer), the signals generated by added labeled nucleobases to each primer may be distinguished. For example, if half of the index primers have a blocked site, then only half of the index primers will be labeled in any SBS sequencing reaction. This reduction in the percentage of index primers which are labeled will lead to a proportional reduction in the intensity of the wavelength of light given off by the index primers in each cluster. By reviewing not only the wavelength of light, but also the intensity of that light from each cluster, the labeled nucleobase attached to the index primer can be distinguished from the labeled nucleobase added to the read primer. This will be discussed more completely in the sections below.

It should be realized that while the examples below may show that some of the index primers are blocked to reduce the intensity of the fluorescent signals received from the nucleobases added to the index primers, any mechanism for differentiating the intensity of the fluorescent labels added to the either the index primer or the read primers are envisioned to be within the scope of the invention. For example, in one aspect the index primers are not blocked, but a proportion or percentage of the read primers contain blocking groups such that labeled nucleobases cannot be added to the read primers. In some embodiments, blocked index primers or blocked read primers may have an irreversibly blocked 3′-end. For example, the blocked primers may be nucleic acids ending with a dideoxynucleotide, or ddNTP (e.g., ddGTP, ddATP, ddTTP or ddCTP), which lacks the 3′ OH group necessary for further growing the nucleic acid chains.

In some embodiments, the disclosed technology comprises obtaining sequence information using Illumina’s sequencing-by-synthesis and reversible terminator-based sequencing chemistry with removable fluorescent dyes (e.g., as described in Bentley et al., Nature 6:53-59 [2009]). Short sequence reads of about tens to a few hundred base pairs may be aligned against a reference genome and unique mapping of the short sequence reads to the reference genome may be identified. After completion of a first, forward sequencing, the templates can be regenerated in situ to enable a second, reverse sequencing from the complementary strands of the templates. Thus, either single-end or paired-end sequencing of the DNA fragments can be used. Detailed information about paired-end sequencing can be found in U.S. Pat. No. 7,601,499 and U.S. Pat. Publication No. 2012/0053063, which are incorporated by reference. Further details regarding the sequencing-by-synthesis and dye labeling methods which can be used by the disclosed technology are described in U.S. Pat. Application Publication Numbers 2007/0166705, 2006/0188901, 2006/0240439, 2006/0281109, 2005/0100900, 2013/0079232, U.S. Pat. Number 7,057,026, PCT Application Publication Numbers WO 2005/065814, WO 2006/064199, WO 2007/010251, and WO 2018/165099, and U.S. Pat. Application Number 17/338590, the disclosures of which are incorporated herein by reference in their entireties.

### Example Sequencer

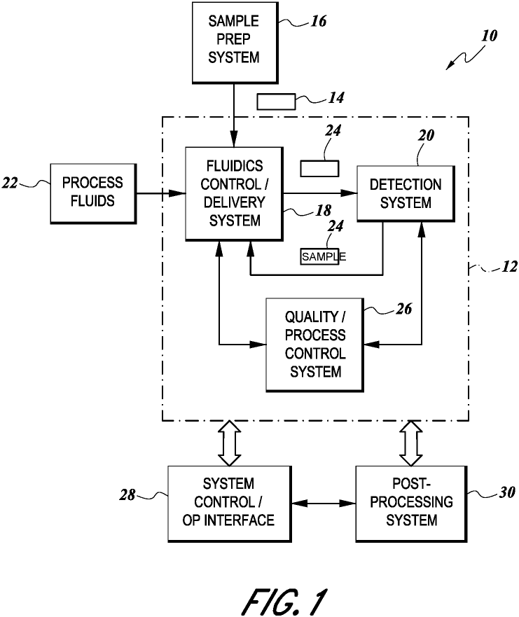

Referring to FIG. 1, a diagrammatical representation of an example sequencing system 10 is illustrated as including a sequencer 12 designed to determine sequences of genetic material of a sample 14. The sequencer may function in a variety of manners, and based upon a variety of techniques, including sequencing by primer extension using labeled nucleotides, as in a presently contemplated embodiment, as well as other sequencing techniques such as sequencing by ligation or pyrosequencing. In some embodiments, the sequencer 12 progressively moves samples through reaction cycles and imaging cycles to progressively build oligonucleotides by binding nucleotides to templates at individual sites on the sample. In some embodiments, the sample may be prepared by a sample preparation system 16. This process may include amplification of fragments of DNA or RNA on a support to create a multitude of sites of DNA or RNA fragments the sequence of which are determined by the sequencing process. Exemplary methods for producing sites of amplified nucleic acids suitable for sequencing include, but are not limited to, rolling circle amplification (RCA) (Lizardi et al., Nat. Genet. 19:225-232 (1998)), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11 (2005); or U.S. Pat. No. 5,641,658), polony generation (Mitra et al., Proc. Natl. Acad. Sci. USA 100:5926-5931 (2003); Mitra et al., Anal. Biochem. 320:55-65 (2003)), or clonal amplification on beads using emulsions (Dressman et al., Proc. Natl. Acad. Sci. USA 100:8817-8822 (2003)) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002)), each of the aforementioned publications is incorporated herein by reference. The sample preparation system 16 may dispose the sample, which may be in the form of an array of sites, in a sample container for processing and imaging.

In some embodiments, the sequencer 12 includes a fluidics control/delivery system 18 and a detection system 20. The fluidics control/delivery system 18 may receive a plurality of process fluids as indicated by reference numeral 22, for circulation through the sample containers of the samples in process, designated by reference numeral 24. As will be appreciated by those skilled in the art, the process fluids may vary depending upon the particular stage of sequencing. For example, in sequencing-by-synthesis (SBS) using labeled nucleotides, the process fluids introduced to the sample may include a polymerase and tagged nucleotides of the four common DNA types, each nucleotide having a unique fluorescent tag and a blocking agent linked to it. The fluorescent tag allows the detection system 20 to detect which nucleotides were last added to primers hybridized to template nucleic acids at individual sites in the array, and the blocking agent prevents addition of more than one nucleotide per cycle at each site.

At other phases of the sequencing cycles, the process fluids 22 may include other fluids and reagents, such as reagents for removing extension blocks from nucleotides or cleaving nucleotide linkers to release a newly extendable primer terminus. For example, once reactions have taken place at individual sites in the array of the samples, the initial process fluid containing the tagged nucleotides may be washed from the sample in one or more flushing operations. The sample may then undergo detection, such as by the optical imaging at the detection system 20. Subsequently, reagents may be added by the fluidics control/delivery system 18 to de-block the last added nucleotide and remove the fluorescent tag from each. The fluidics control/delivery system 18 may then again wash the sample, which is then prepared for a subsequent cycle of sequencing. Exemplary fluidic and detection configurations that can be used in the methods and devices set forth herein are described in WO 07/123744, which is incorporated herein by reference. In some embodiments, such sequencing may continue until the quality of data derived from sequencing degrades due to cumulative loss of yield or until a predetermined number of cycles have been completed.

In some embodiments, the quality of samples 24 in process as well as the quality of the data derived by the system, and the various parameters used for processing the samples is controlled by a quality/process control system 26. The quality/process control system 26 may include one or more programmed processors, or general purpose or application-specific computers which communicate with sensors and other processing systems within the fluidics control/delivery system 18 and the detection system 20. A number of process parameters may be used for sophisticated quality and process control, for example, as part of a feedback loop that can change instrument operation parameters during the course of a sequencing run.

In some embodiments, the sequencer 12 also communicates with a system control/operator interface 28 and ultimately with a post-processing system 30. The system control/operator interface 28 may include a general purpose or application-specific computer designed to monitor process parameters, acquired data, system settings, and so forth. The operator interface may be generated by a program executed locally or by programs executed within the sequencer 12. In some embodiments, these may provide visual indications of the health of the systems or subsystems of the sequencer, the quality of the data acquired, and so forth. The system control/operator interface 28 may also permit human operators to interface with the system to regulate operation, initiate and interrupt sequencing, and any other interactions that may be desired with the system hardware or software. For instance, the system control/operator interface 28 may automatically undertake and/or modify steps to be performed in a sequencing procedure, without input from a human operator. Alternatively or additionally, the system control/operator interface 28 may generate recommendations regarding steps to be performed in a sequencing procedure and display these recommendations to the human operator. This mode may allow for input from the human operator before undertaking and/or modifying steps in the sequencing procedure. In addition, the system control/operator interface 28 may provide an option to the human operator allowing the human operator to select certain steps in a sequencing procedure to be automatically performed by the sequencer 12 while requiring input from the human operator before undertaking and/or modifying other steps. In any event, allowing both automated and operator interactive modes may provide increased flexibility in performing the sequencing procedure. In addition, the combination of automation and human-controlled interaction may further allow for a system capable of creating and modifying new sequencing procedures and algorithms through adaptive machine learning based on the inputs gathered from human operators.

The post-processing system 30 may further include one or more programmed computers that receive detected information, which may be in the form of pixilated image data and derive sequence data from the image data. The post-processing system 30 may include image recognition algorithms which distinguish between colors of dyes (e.g., fluorescent emission spectra of dyes) attached to nucleotides that bind at individual sites as sequencing progresses (e.g., by analysis of the image data encoding specific colors and/or intensities), and logs the sequence of the nucleotides at the individual site locations. Progressively, then, the post-processing system 30 may build sequence lists for the individual sites of the sample array which can be further processed to establish genetic information for extended lengths of material by various bioinformatics algorithms.

The sequencing system 10 may be configured to handle individual samples or may be designed for higher throughput in a manner in which multiple stations are provided for the delivery of reagents and other fluids, and for detection of progressively building sequences of nucleotides. Further details can be found in U.S. Pat. No. 9797012, which is incorporated herein by reference.

Samples may be removed from processing, reprocessed, and scheduling of such processing may be altered in real time, particularly where the fluidics control system 18 or the quality/process control system 26 detect that one or more operations were not performed in an optimal or desired manner. In embodiments wherein a sample is removed from the process or experiences a pause in processing that is of a substantial duration, the sample can be placed in a storage state. Placing the sample in a storage state can include altering the environment of the sample or the composition of the sample to stabilize biomolecule reagents, biopolymers or other components of the sample. Exemplary methods for altering the sample environment include, but are not limited to, reducing temperature to stabilize sample constituents, addition of an inert gas to reduce oxidation of sample constituents, and removing from a light source to reduce photobleaching or photodegradation of sample constituents. Exemplary methods of altering sample composition include, without limitation, adding stabilizing solvents such as antioxidants, glycerol and the like, altering pH to a level that stabilizes enzymes, or removing constituents that degrade or alter other constituents. In addition, certain steps in the sequencing procedure may be performed before removing the sample from processing. For instance, if it is determined that the sample should be removed from processing, the sample may be directed to the fluidics control/delivery system 18 so that the sample may be washed before storage. These steps may be taken to ensure that no information from the sample is lost.

Moreover, sequencing operations may be interrupted by the sequencer 12 at any time upon the occurrence of certain predetermined events. These events may include, without limitation, unacceptable environmental factors such as undesirable temperature, humidity, vibrations or stray light; inadequate reagent delivery or hybridization; unacceptable changes in sample temperature; unacceptable sample site number/quality/distribution; decayed signal-to-noise ratio; insufficient image data; and so forth. It should be noted that the occurrence of such events need not require interruption of sequencing operations. Rather, such events may be factors weighed by the quality/process control system 26 in determining whether sequencing operations should continue. For example, if an image of a particular cycle is analyzed in real time and shows a low signal for that optical channel, the image can be re-exposed using a longer exposure time, or have a particular chemical treatment repeated. If the image shows a bubble in a flow cell, the instrument can automatically flush more reagent to remove the bubble, then re-record the image. If the image shows low signal for a particular optical channel in one cycle due to a fluidics problem, the instrument can automatically halt scanning and reagent delivery for that particular optical channel, thus saving on analysis time and reagent consumption.

Although the system has been exemplified above with regard to a system in which a sample interfaces with different stations by physical movement of the sample, it will be understood that the principles set forth herein are also applicable to a system in which the steps occurring at each station are achieved by other means not requiring movement of the sample. For example, reagents present at the stations can be delivered to a sample by means of a fluidic system connected to reservoirs containing the various reagents. Similarly, an optics system can be configured to detect a sample that is in fluid communication with one or more reagent stations. Thus, detection steps can be carried out before, during or after delivery of any particular reagent described herein. Accordingly, samples can be effectively removed from processing by discontinuing one or more processing steps, be it fluid delivery or optical detection, without necessarily physically removing the sample from its location in the device.

Disclosed systems can be used to continuously sequence nucleic acids in a plurality of different samples. Disclosed systems can be configured to include an arrangement of samples and an arrangement of stations for carrying out sequencing steps. The samples in the arrangement of samples can be placed in a fixed order and at fixed intervals relative to each other. For example, an arrangement of nucleic acid arrays can be placed along the outer edge of a circular table. Similarly, the stations can be placed in a fixed order and at fixed intervals relative to each other. For example, the stations can be placed in a circular arrangement having a perimeter that corresponds to the layout for the arrangement of sample arrays. Each of the stations can be configured to carry out a different manipulation in a sequencing protocol. The arrangements of sample arrays and stations can be moved relative to each other such that the stations carry out desired steps of a reaction scheme at each reaction site. The relative locations of the stations and the schedule for the relative movement can correlate with the order and duration of reaction steps in the sequencing reaction scheme such that once a sample array has completed a cycle of interacting with the full set of stations, then a single sequencing reaction cycle is complete. For example, primers that are hybridized to nucleic acid targets on an array can each be extended by addition of a single nucleotide, detected and de-blocked if the order of the stations, spacing between the stations, and rate of passage for the array corresponds to the order of reagent delivery and reaction time for a complete sequencing reaction cycle.

In accordance with the configuration set forth above, each lap (or full revolution in embodiments where a circular table is used) completed by an individual sample array can correspond to determination of a single nucleotide for each of the target nucleic acids on the array (e.g., including the steps of incorporation, imaging, cleavage and de-blocking carried out in each cycle of a sequencing run). Furthermore, several sample arrays present in the system (for example, on the circular table) concurrently move along similar, repeated laps through the system, thereby resulting in continuous sequencing by the system. Using the disclosed systems or methods, reagents can be actively delivered or removed from a first sample array in accordance with a first reaction step of a sequencing cycle while incubation, or some other reaction step in the cycle, occurs for a second sample array. Thus, a set of stations can be configured in a spatial and temporal relationship with an arrangement of sample arrays such that reactions occur at multiple sample arrays concurrently even as the sample arrays are subjected to different steps of the sequencing cycle at any given time, thereby allowing continuous and simultaneous sequencing to be performed. Such a circular system may be used when the chemistry and imaging times are disproportionate. For small flow cells that only take a short time to scan, the system may have a number of flow cells running in parallel in order to optimize the time the instrument spends acquiring data. When the imaging time and chemistry time are equal, a system that is sequencing a sample on a single flow cell spends half the time performing a chemistry cycle rather than an imaging cycle, and therefore a system that can process two flow cells could have one on the chemistry cycle and one on the imaging cycle. When the imaging time is ten-fold less than the chemistry time, the system can have ten flow cells at various stages of the chemistry process whilst continually acquiring data.

In some embodiments, the disclosed system is configured to allow replacement of a first sample array with a second sample array while the system continuously sequences nucleic acids of a third sample array. Thus, a first sample array can be individually added or removed from the system without interrupting sequencing reactions occurring at another sample array, thereby allowing continuous sequencing for the set of sample arrays. Moreover, sequencing runs of different lengths can be performed continuously and simultaneously in the system because individual sample arrays can complete a different number of laps through the system and the sample arrays can be removed or added to the system in an independent fashion such that reactions occurring at other sites are not perturbed.

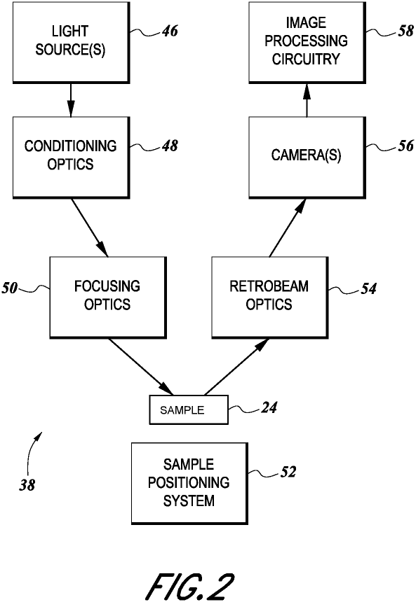

FIG. 2 illustrates an exemplary detection station 38 which can detect nucleotides added at sites of an array and can be used in conjunction with the example sequencing system of FIG. 1. As set forth above, a sample can be moved to two or more stations of the device that are located in physically different locations or alternatively one or more steps can be carried out on a sample that is in communication with the one or more stations without necessarily being moved to different locations. Accordingly, the description herein with regard to particular stations is understood to relate to stations in a variety of configurations whether or not the sample moves between stations, the stations move to the sample, or the stations and sample are static with respect to each other. In the embodiment illustrated in FIG. 2, one or more light sources 46 provide light beams that are directed to conditioning optics 48. The light sources 46 may include one or more lasers, with multiple lasers being used for detecting dyes that fluoresce at different corresponding wavelengths. The light sources may direct beams to the conditioning optics 48 for filtering and shaping of the beams in the conditioning optics. For example, in a presently contemplated embodiment, the conditioning optics 48 combine beams from multiple lasers and generate a substantially linear beam of radiation that is conveyed to focusing optics 50. The laser modules can additionally include a measuring component that records the power of each laser. The measurement of power may be used as a feedback mechanism to control the length of time an image is recorded in order to obtain a uniform exposure energy, and therefore signal, for each image. If the measuring component detects a failure of the laser module, then the instrument can flush the sample with a “holding buffer” to preserve the sample until the error in the laser can be corrected.

The sample 24 is positioned on a sample positioning system 52 that may appropriately position the sample in three dimensions and may displace the sample for progressive imaging of sites on the sample array. In a presently contemplated embodiment, the focusing optics 50 confocally direct radiation to one or more surfaces of the array at which individual sites are located that are to be sequenced. Depending upon the wavelengths of light in the focused beam, a retrobeam of radiation is returned from the sample due to fluorescence of dyes bound to the nucleotides at each site.

The retrobeam is then returned through retrobeam optics 54 which may filter the beam, such as to separate different wavelengths in the beam, and direct these separated beams to one or more cameras 56. The cameras 56 may be based upon any suitable technology, such as including charge coupled devices that generate pixilated image data based upon photons impacting locations in the devices. In some embodiments, the cameras 56 may include CMOS sensors. In some embodiments, the cameras 56 may include one or more point-and-shoot cameras. In some embodiments, the cameras 56 may include one or more time delay and integration (TDI) cameras. The cameras generate image data that is then forwarded to image processing circuitry 58. In some embodiments, the processing circuitry 58 may perform various operations, such as analog-to-digital conversion, scaling, filtering, and association of the data in multiple frames to appropriately and accurately image multiple sites at specific locations on the sample. The image processing circuitry 58 may store the image data and may ultimately forward the image data to the post-processing system 30 where sequence data can be derived from the image data. Example detection devices that can be used at a detection station include, for example, those described in US 2007/0114362 (U.S. Pat. Application Ser. No. 11/286,309) and WO 07/123744, each of which is incorporated herein by reference.

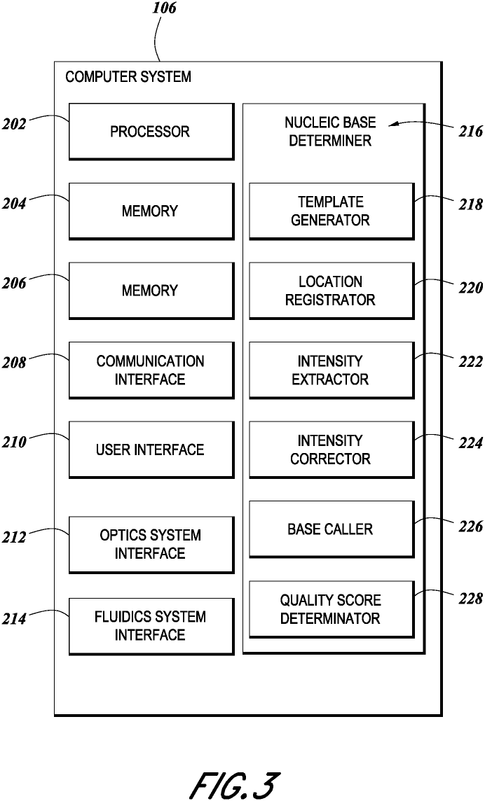

A computer system 106 as illustrated in FIG. 3 may be used to implement the system control/operator interface 28 and the post-processing system 30 of the example sequencing system 10 in FIG. 1. As shown in FIG. 3, the computer system 106 can include functionalities for controlling optics/fluidics systems and determining nucleobase sequences of polynucleotides.

In one embodiment, the computer system 106 includes a processor 202 that is in electrical communication with a memory 204, a storage 206, and a communication interface 208. The processor 202 can be configured to execute instructions that cause the fluidics system 104 to supply reagents to the flow cell 114 during sequencing reactions. The processor 202 can execute instructions that control the light source 120 of the optics system 102 to generate light at around a predetermined wavelength. The processor 202 can execute instructions that control the detector 126 of the optics system 102 and receive data from the detector 126. The processor 202 can execute instructions to process data, for example fluorescent images, received from the detector 126 and to determine the nucleotide sequences of polynucleotides based on the data received form the detector 126. The memory 204 can be configured to store instructions for configuring the processor 202 to perform the functions of the computer system 106 when the sequencing system 100 is powered on. When the sequencing system 100 is powered off, the storage 206 can store the instructions for configuring the processor 202 to perform the functions of the computer system 106. The communication interface 208 can be configured to facilitate the communications between the computer system 106, the optics system 102, and the fluidics system 104.

The computer system 106 can include a user interface 210 configured to communicate with a display device (not shown) for displaying the sequencing results of the sequencing system 100. The user interface 210 can be configured to receive inputs from users of the sequencing system 100. An optics system interface 212 and a fluidics system interface 214 of the computer system 106 can be configured to control the optics system 102 and the fluidics system 104 through the communication links 108a and 108b illustrated in FIG. 1A. For example, the optics system interface 212 can communicate with the computer interface 110 of the optics system 102 through the communication link 108a.

The computer system 106 can include a nucleic base determiner 216 configured to determine the nucleotide sequence of polynucleotides using the data received from the detector 126. The nucleic base determiner 216 can include one or more of: a template generator 218, a location registrator 220, an intensity extractor 222, an intensity corrector 224, a base caller 226, and a quality score determiner 228. The template generator 218 can be configured to generate a template of the locations of polynucleotide clusters in the flow cell 114 using the fluorescent images captured by the detector 126. The location registrator 220 can be configured to register the locations of polynucleotide clusters in the flow cell 114 in the fluorescent images captured by the detector 126 based on the location template generated by the template generator 218. The intensity extractor 222 can be configured to extract intensities of the fluorescent emissions from the fluorescent images to generate extracted intensities. For example, the peak intensity value found in a diffraction-limited spot of a DNA cluster may be extracted from the image and used to represent the signal of the DNA cluster. For another example, the total intensity included within a diffraction-limited spot of a DNA cluster may be extracted from the image and used to represent the signal of the DNA cluster. Alternatively, the intensity estimate can be made through the use of equalization and channel estimation.

The intensity corrector 224 can be configured to reduce or eliminate noise or aberration inherent in the sequencing reaction or optical system. For example, intensity may be influenced by laser intensity fluctuation, DNA cluster shape/size variation, uneven illumination, optical distortions or aberrations, and/or phasing/pre-phasing that occur in the DNA clusters. In some embodiments, the intensity corrector 224 can phase correct or pre-phase correct extracted intensities. The base caller 226 can be configured to determine the nucleobases of a polynucleotide from the corrected intensities. The bases of a polynucleotide determined by the base caller 226 can be associated with quality scores determined by the quality score determiner 228. Quality scoring refers to the process of assigning a quality score to each base call. To evaluate the quality of a base call from a sequencing read, example processes can include calculating a set of predictor values for the base call and using the predictor values to look up a quality score in a quality table. The quality score can be presented in any suitable format that allows a user to determine the probability of error of any given base call. In some embodiments, the quality score is presented as a numerical value. For example, the quality score can be quoted as QXX where the XX is the score and it means that that particular call has a probability of error of 10^{-XX/10}. Thus, as an example, Q30 equates to an error rate of 1 in 1000, or 0.1% and Q40 equates to an error rate of 1 in 10,000 or 0.01%. The error rate can be calculated using a control nucleic acid. Additionally, some metrics displays can include the error rate on a per-cycle basis. In some embodiments, the quality table is generated using on a calibration data set, the calibration set being representative of run and sequence variability. Further details of the computations that can be performed by the nucleic base determiner, calculation of error rate and quality score may be found in U.S. Pat. Number 8392126, U.S. Pat. Application Publication Numbers 2020/0080142 and 2012/0020537, each of which is incorporated by reference herein in its entirety.

### Parallel Insert and Index Sequencing

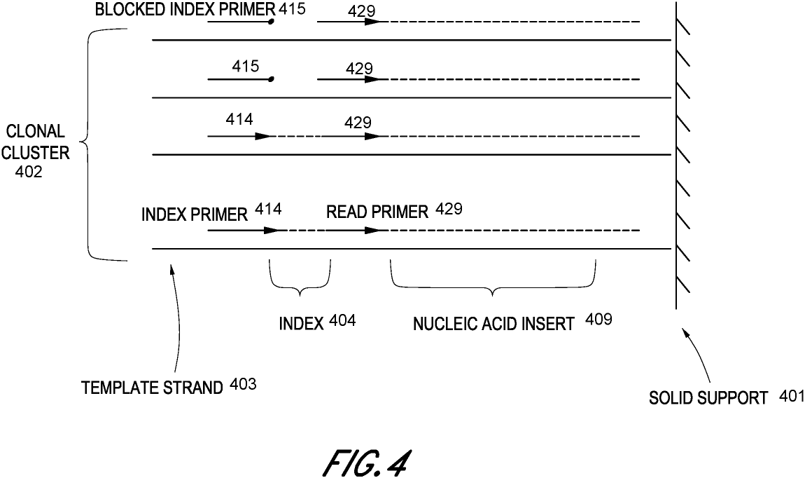

FIG. 4 shows a schematic of sequencing an indexed sample according to one embodiment of the disclosed technology. A clonal nucleic acid cluster 402 may be one of many clonal nucleic acid clusters that are attached to the surface of a solid support 401 (e.g., a part of a flow cell). The clonal nucleic acid cluster 402 may include clonal copies of a template polynucleotide 403. For example, the clonal nucleic acid cluster 402 may be generated by bridge PCR amplification of a template polynucleotide. In some embodiments, the template polynucleotide may be an indexed nucleic acid sample. For example, the template polynucleotide strand 403 may include an insert portion 409 which is derived from a nucleic acid sample and an index portion 404 which is added during a library preparation process. The added index portion may include a barcode which uniquely identifies the sample (e.g., uniquely indicates the source and/or the batch number of the sample). The added index portion may be a nucleic acid of about 6, 10, 15, 20, 25, or any value therebetween, nucleotides in length.

The library preparation process may also add a primer binding site next to the insert portion 409, such that a read primer 429 can specifically hybridize next to the insert portion 409 to initiate polymerization/primer extension reaction and allow sequencing of the insert portion 409 as fluorescently labeled nucleotides are added to the extended read primers. Similarly, the library preparation process may add another primer binding site next to the index portion 404, such that an index primer 414 can specifically hybridize next to the index portion 404 to initiate polymerization/primer extension reaction and allow sequencing of the index portion 404 as fluorescently labeled nucleotides are added to the extended index primers. However, since the fluorescent signal emitted by the extended read primers and the fluorescent signal emitted by the extended index primers are co-located and may not be optically resolved, methods for determining whether a fluorescent signal is associated with the extended read primers or the extended index primers is needed, at least when the labeled nucleotides at the extended read primers are not the same as the labeled nucleotides at the extended index primers (e.g., when “A”s are added to the read primers and “C”s are added to the index primers), in order to correctly determine the nucleic acid sequences of the insert portion 409 and the index portion 404. One way to determine whether a fluorescent signal is associated with the extended read primers or the extended index primers is to use distinguishable levels of signal intensity. In some embodiments, a fraction (e.g., one fourth, one third, half, two thirds, etc., or any value therebetween) of the index primer is chemically blocked and cannot be extended by polymerase. Blocked index primers are labeled as reference number 415 in FIG. 4. For example, a fraction of index primers may have a chemically blocked 3′-end, while all the read primers 429 may have an unblocked 3′-end. As a result, the number of index primers that are extended in the cluster may be smaller compared to that of the read primers; the number of labeled nucleotides associated with extended index primers in the cluster may be smaller compared to that of the read primers. After receiving fluorescent excitation, the signal emitted from the labeled nucleotides added to index primers may be weaker compared to that of the read primers. Thus, the weaker signal and the nucleotide identity it represents can be determined as associating with extended index primers.

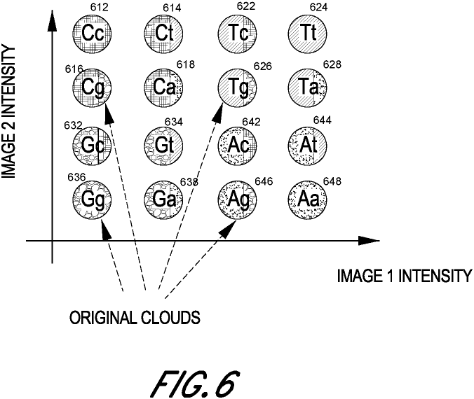

In use, read primers, blocked index primers and unblocked index primers may be mixed together, provided to the flow cell as a mixture, and hybridized to the template polynucleotide strands (e.g., hybridized simultaneously/in the same reaction step). Then, sequencing-by-synthesis reaction cycles may be performed with fluorescently labeled nucleotide analogs, for example. Therefore, the fluorescent signal emitted from the cluster 402 may come from both the extended read primers 429 and the extended unblocked index primers 414. Moreover, the intensity of the fluorescent signals emitted from the collection of extended read primers 429 and the collection of extended unblocked index primers 414 in the cluster may be proportional to the amount of the two types primer molecules in the cluster, or the amount of labeled nucleotide analogs on the two types of extended primer molecules. Thus, a determination of whether a signal comes from the extended read primers 429 or from the extended unblocked index primers 414 can be made based on the intensity (or brightness) of the received signal. For example, if half of the index primers are blocked, the intensity of the signal from the labeled nucleotides added to the index portion 404 will be half as strong compared to the intensity of the signal from the labeled nucleotides added to the insert portion 409. Therefore, both the nucleobase at the index portion 404 and the nucleobase at the insert portion 409 can be identified in parallel (or substantially simultaneously), provided that the signal-to-noise ratio (SNR) in the sequencing system remains sufficiently high (above a threshold of about 8 dB, 10 dB, or 12 dB, for example) to distinguish the half-intensity signals from the full-intensity signals. (That is, the bins shown in FIG. 6 are well separated and can be resolved, for example.) In this stage of simultaneous identification of two nucleobases in a nucleic acid cluster, the SNR may be calculated as the mean base-called intensity divided by standard deviation of non-based-called intensities, where the intensities are associated with two signals coming from each cluster. In one aspect, considering the sixteen bins in FIG. 6, for example, the SNR may be calculated as the difference in mean intensities between two bins divided by the standard deviation of the intensities in those bins, where the sixteen bins represent distribution of intensities associated with two signals coming from each cluster. Over the course of SBS reaction cycles, the SNR in the sequencing system may decay due to issues such as strand termination, polymerase damage, etc. Therefore, a SNR that is much higher than what is sufficient to achieve a required base-calling error rate (e.g., 0.01%) may be used at the beginning of the course of SBS reaction cycles. For example, a SNR above 20 dB may be used at the beginning of a sequencing run, and the SNR may gradually decay over SBS cycles, reaching the threshold (e.g., 8 dB, 10 dB, or 12 dB) after about 10 or 20 SBS cycles. Thus, the surplus of SNR at the beginning of the sequencing run may allow for simultaneous identification of the nucleobase at the index portion 404 and the nucleobase at the insert portion 409 up to about 10 or 20 SBS cycles.

The embodiment of using a fraction of blocked primers for the index portion and unblocked primers for the insert portion can help ensure that the index signal (the signal from the index portion 404) and the insert signal (the signal from the insert portion 409) are physically collocated at the same cluster location. Further, it can ensure that the ratio of the index-to-insert signal (i.e., the signal from the index portion 404 to the signal from the insert portion 409) is maintained on a per-cluster basis, and thus there is no expected impact to the ratio of the signals from the size of the cluster. That is, whether a cluster is bigger or smaller (e.g., contains more or fewer copies of the template strand), its insert signal and index signal will both be scaled by the same amount according to the cluster size.

In some embodiments, after a certain number of primer extension steps/sequencing cycles (e.g., about 10 or 20 cycles), or if the signal-to-noise ratio in the sequencing system drops below the desired threshold (e.g., 8 dB, 10 dB, or 12 dB), the index primers may be removed from the cluster 402 such that sequencing only occurs from the read primers 429. For example, the index primer may be thermally de-hybridized. As another example, the index primer may be removed by contacting a locked nucleic acid (LNA) with the template polynucleotide strands to displace the index primer, and the LNA may be chemically blocked and may not polymerize. In some embodiments, the extended index primers may eventually run into the read primers 429 and polymerization from the index primers 414 may stop. In one example, a non-strand displacing polymerase may be used so that extension of the index primer stops when the extended portion contacts the hybridized read primer. In some embodiments, the extended index primers may eventually run into the solid support 401 and polymerization from the index primers 414 may stop (for example, in a scenario where polymerization runs towards the solid support 401 but the index portion 404 is closer to the solid support compared to the insert portion 409). After removal of the index primers or after polymerization from the index primers stops, reaction from the read primers 429 may continue and the insert portion 409 may continue to be sequenced. At the stage of identifying only one nucleobase in a nucleic acid cluster (sequencing only the insert portion 409, e.g., after about 10 or 20 cycles), the signal-to-noise ratio (SNR) in the sequencing system may be calculated differently, for example as the mean base-called intensity divided by a standard deviation of non-based-called intensities, where the intensities are associated with only one signal from each cluster. In one aspect, considering only four bins in a scatter plot of distribution of signals from a nucleic acid cluster, for example as described in U.S. Publication No. 2013/0079232, which is incorporated herein by reference. In this scenario, the SNR may be calculated as the difference in mean intensities between two bins divided by the standard deviation of the intensities in those bins, where the four bins represent distribution of intensities associated with only one signal from each cluster. The SNR during the stage of identifying only one nucleobase in a cluster may gradually decay over sequencing cycles due to similar issues as mentioned above, reaching a threshold (e.g., 8 dB, 10 dB, or 12 dB, corresponding to a minimally required base-calling error rate) after about 500 SBS cycles.

Accordingly, the disclosed technology provides a more efficient sequencing workflow in that the index primers and the read primers can be hybridized and/or de-hybridized (upon completion of sequencing the template strand) in the same operation (or substantially simultaneously), thus saving time compared to using separate operations. Further, the disclosed technology provides a more efficient sequencing workflow in that the index portion can be sequenced in parallel (or simultaneously) with the insert portion, hence saving read cycles of SBS compared to sequencing the index portion and the insert portion separately (or in series). Saving read cycles may allow the sequencing workflow to be implemented with a shorter run time, e.g., the run time saving may be about half an hour, in some examples.

In further embodiments of the disclosed technology, the ratio of the index primers and the read primers bound to the cluster may be adjusted and determined prior to sequencing, and this information may be used to calibrate the fluorescent imaging data obtained during sequencing. For example, the ratio of the index primers and the read primers hybridized to the cluster may be monitored by fluorescent imaging if different fluorophores are attached to the index primers and the read primers. The index primers and the read primers may be removed at different rates by regulated thermal de-hybridization until a desired ratio is achieved. Alternatively, the fluorophores may be chemically removed from the index primers and the read primers prior to sequencing.

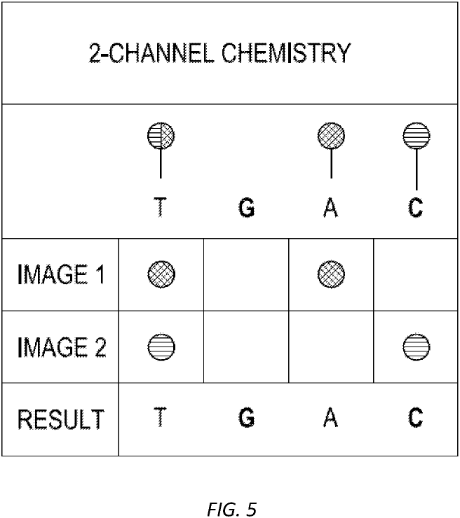

FIG. 5 shows an example dye labeling scheme that may be used in conjunction with the embodiment of the disclosed technology illustrated in FIG. 4. As shown in FIG. 5, different types of nucleotide analogs may be labeled by different fluorescent labels/dyes having different absorption and/or emission spectra. For example, dGTP is unlabeled, dATP is labeled with a first label/dye, dCTP is labeled with a second label/dye, and dTTP is labeled with a third label/dye, and the different types of nucleotide analogs may thus produce distinct characteristics of fluorescence emissions after being excited by a light source. Alternative dye labeling schemes may be contemplated. For example, dTTP may be labeled with two different dyes. In some embodiments, the absorption spectra of the dyes allow them to be excited by a single light source at a predetermined wavelength, such as a “blue” laser at about 450 nm. However, embodiments are not limited to light sources generating this particular wavelength of light, and other wavelengths corresponding to red, green, violet or other available wavelengths of light are contemplated. In other embodiments, two or more light sources may be used to excite the dyes if the absorption spectra of the dyes are sufficiently different.

The first fluorescent label/dye may have an emission spectrum that can be captured in a first image taken in a first optical channel (e.g., “IMAGE 1” in FIG. 5). The second fluorescent label/dye may have an emission spectrum that can be captured in a second image taken in a second optical channel (e.g., “IMAGE 2” in FIG. 5) which is distinct from the first optical channel. The third fluorescent label/dye may have an emission spectrum that can be captured in both the first and second optical channels (e.g., both “IMAGE 1” and “IMAGE 2” in FIG. 5). As a result, in the example shown in FIG. 5, dTTP can be identified as showing in both the first and second images with sufficiently high intensities. dGTP may show as having very low intensities (e.g., lower than a cutoff value) in either images. dATP can be identified as showing in the first image with a sufficiently high intensity but a very low intensity (e.g., lower than a cutoff value) in the second image. dCTP can be identified as showing in the second image with a sufficiently high intensity but a very low intensity (e.g., lower than a cutoff value) in the first image. The fluorescent dyes conjugated to the four types of nucleotide analogs are illustrative only, and not intended to be limiting. In other embodiments, the nucleotide analog not conjugated with any fluorescent dye may be dTTP, dCTP, or dATP. In other embodiments, the nucleotide analog conjugated with the first fluorescent dye may be dGTP, dCTP, or dTTP. In other embodiments, the nucleotide analog conjugated with the second fluorescent dye may be dGTP, dTTP, or dATP. In other embodiments, the nucleotide analog conjugated the third fluorescent dye may be dGTP, dATP, or dCTP.

In some embodiments, the nucleotide analogs used in the disclosed sequencing system may be fully functionalized nucleotides. The linkers located between the nucleotide base and the fluorescent molecule may include one or more cleavage groups. Prior to the subsequent sequencing cycle, the fluorescent labels can be removed from the nucleotide analogs by cleavage of the linker. For example, a linker attaching a fluorescent label to a nucleotide analog can include an azide and/or an alkoxy group, for example on the same carbon, such that the linker may be cleaved after each incorporation cycle by a phosphine reagent, thereby releasing the fluorescent label. The nucleotide triphosphates can be reversibly blocked at the 3′ position so that sequencing is controlled, and no more than a single nucleotide analog can be added onto each extending primer-polynucleotide in each cycle. For example, the 3′ ribose position of a nucleotide analog can include both alkoxy and azido functionalities which can be removable by cleavage with a phosphine reagent, thereby creating a nucleotide that can be further extended. Prior to the subsequent sequencing cycle, the reversible 3′ blocks can be removed so that another nucleotide analog can be added onto each extending primer-polynucleotide.

In some embodiments, the fluorescent labels are selected from the group consisting of polymethine derivatives, coumarin derivatives, benzopyran derivatives, chromenoquinoline derivatives, compounds containing bis-boron heterocycles such as BOPPY and BOPYPY. In some embodiments, the fluorescent label is attached to the nucleotide through a cleavable linker. In some further embodiments, the labeled nucleotide may have the fluorescent label attached to the C5 position of a pyrimidine base or the C7 position of a 7-deaza purine base, optionally through a cleavable linker moiety. For example, the nucleobase may be 7-deaza adenine and the dye is attached to the 7-deaza adenine at the C7 position, optionally through a cleavable linker. The nucleobase may be 7-deaza guanine and the dye is attached to the 7-deaza guanine at the C7 position, optionally through a cleavable linker. The nucleobase may be cytosine and the dye is attached to the cytosine at the C5 position, optionally through a cleavable linker. As another example, the nucleobase may be thymine or uracil and the dye is attached to the thymine or uracil at the C5 position, optionally through a cleavable linker. In some further embodiments, the cleavable linker may comprise similar or the same chemical moiety as the reversible terminator 3′ hydroxy blocking group such that the 3′ hydroxy blocking group and the cleavable linker may be removed under the same reaction condition or in a single chemical reaction. Non-limiting example of the cleavable linker include the LN3 linker, the sPA linker, and the AOL linker, each of which is exemplified below.

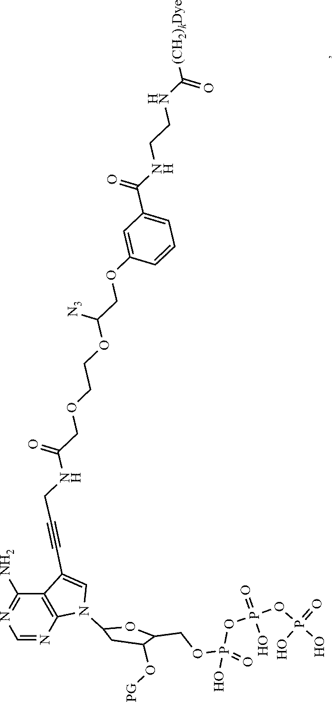

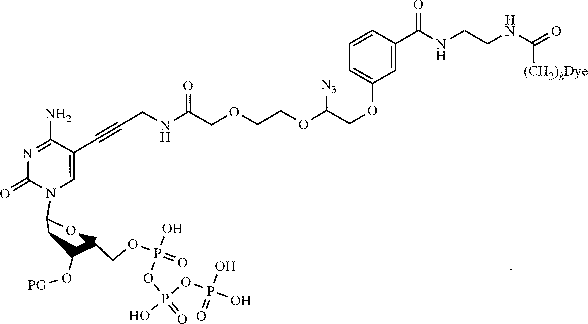

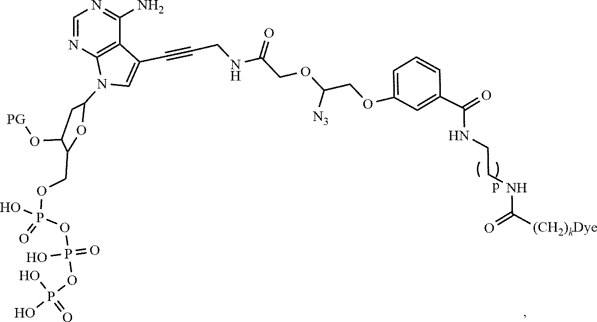

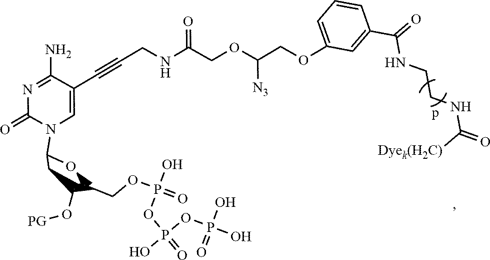

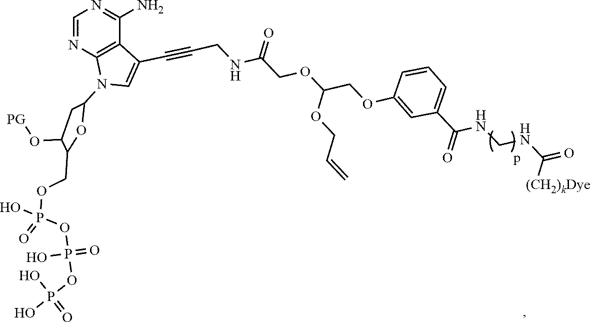

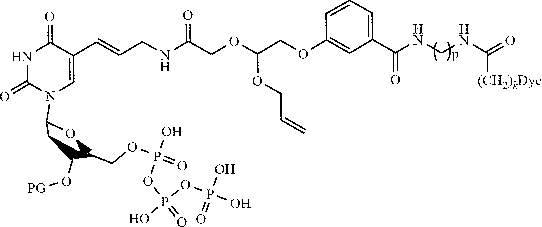

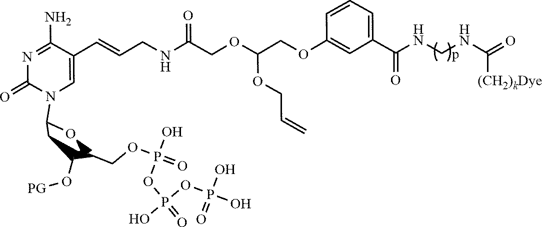

In some embodiments, the nucleotides are selected from the group consisting of an analog of dGTP, an analog of dTTP, an analog of dUTP, an analog of dCTP, and an analog of dATP. In some embodiments, the first nucleotide is a first reversibly blocked nucleotide triphosphate (rbNTP), the second nucleotide is a second rbNTP, the third nucleotide is a third rbNTP, and the fourth nucleotide is a fourth rbNTP, wherein each of the first nucleotide, second nucleotide, third nucleotide and fourth nucleotide is a different type of nucleotide from the other. In some embodiments, the four rbNTPs are selected from the group consisting of rbATP, rbTTP, rbUTP, rbCTP, and rbGTP. In some embodiments, each of the four rbNTPs includes a modified base and a reversible terminator 3′ blocking group. Non-limiting example of the 3′ blocking group include azidomethyl (*—CH₂N₃), substituted azidomethyl (e.g., *—CH(CHF₂)N₃ or *—CH(CH₂F)N₃) and *—CH₂—O—CH₂—CH═CH₂, where the asterisk * indicates the point attachment to the 3′ oxygen of the ribose or deoxyribose ring of the nucleotide.

Further details about the dyes and the fully functionalized nucleotides can be found in U.S. Pat. Application Publication Numbers 2018/0094140 and 2020/0277670, International Patent Application Publication Number 2017/051201, and U.S. Provisional Pat. Application Numbers 63/057758 and 63/127061, the disclosures of which are incorporated herein by reference in their entireties.

FIG. 6 is a scatter plot showing an example of sixteen distributions of signals from a nucleic acid cluster according to the embodiment of the disclosed technology illustrated in FIG. 4, which may be implemented with the dye labeling scheme shown in FIG. 5 in one example. As explained in connection with FIG. 4, in one embodiment, the fluorescent signal coming from the collection of extended read primers 429 will be brighter than the fluorescent signal coming from the collection of extended unblocked index primers 414 in the same cluster. The scatter plot of FIG. 6 shows sixteen distributions (or bins) of intensity values from the combination of a brighter signal and a dimmer signal of a cluster; the two signals may be co-localized and may not be optically resolved. The intensity values shown in FIG. 6 may be up to a scale factor; the units of the intensity values may be arbitrary. The sum of the brighter signal from the extended read primers 429 and the dimmer signal from the extended unblocked index primers 414 results in a combined signal. The combined signal may be captured by the first optical channel and the second optical channel (e.g., the “IMAGE 1” channel and the “IMAGE 2” channel in FIG. 5). Since the brighter signal may be A, T, C or G, and the dimmer signal may be A, T, C or G, there are sixteen possibilities for the combined signal, corresponding to sixteen distinguishable patterns when optically captured according to the embodiment described in connection with FIG. 5. That is, each of the sixteen possibilities corresponds to a bin shown in FIG. 6. The computer system can map the combined signal from a cluster into one of the sixteen bins, and thus determine the added nucleobase at the extended read primers 429 and the added nucleobase at the extended unblocked index primers 414, respectively.

For example, when the combined signal is mapped to bin 612 for a base calling cycle, the computer processor base calls both the added nucleobase at the extended read primers 429 and the added nucleobase at the extended unblocked index primers 414 as C. When the combined signal is mapped to bin 614 for the base calling cycle, the processor base calls the added nucleobase at the extended read primers 429 as C and the added nucleobase at the extended unblocked index primers 414 as T. When the combined signal is mapped to bin 616 for the base calling cycle, the processor base calls the added nucleobase at the extended read primers 429 as C and the added nucleobase at the extended unblocked index primers 414 as G. When the combined signal is mapped to bin 618 for the base calling cycle, the processor base calls the added nucleobase at the extended read primers 429 as C and the added nucleobase at the extended unblocked index primers 414 as A.

When the combined signal is mapped to bin 622 for the base calling cycle, the processor base calls the added nucleobase at the extended read primers 429 as T and the added nucleobase at the extended unblocked index primers 414 as C. When the combined signal is mapped to bin 624 for the base calling cycle, the processor base calls both the added nucleobase at the extended read primers 429 and the added nucleobase at the extended unblocked index primers 414 as T. When the combined signal is mapped to bin 626 for the base calling cycle, the processor base calls the added nucleobase at the extended read primers 429 as T and the added nucleobase at the extended unblocked index primers 414 as G. When the combined signal is mapped to bin 628 for the base calling cycle, the processor base calls the added nucleobase at the extended read primers 429 as T and the added nucleobase at the extended unblocked index primers 414 as A.

When the combined signal is mapped to bin 632 for the base calling cycle, the processor base calls the added nucleobase at the extended read primers 429 as G and the added nucleobase at the extended unblocked index primers 414 as C. When the combined signal is mapped to bin 634 for the base calling cycle, the processor base calls the added nucleobase at the extended read primers 429 as G and the added nucleobase at the extended unblocked index primers 414 as T. When the combined signal is mapped to bin 636 for the base calling cycle, the processor base calls both the added nucleobase at the extended read primers 429 and the added nucleobase at the extended unblocked index primers 414 as G. When the combined signal is mapped to bin 638 for the base calling cycle, the processor base calls the added nucleobase at the extended read primers 429 as G and the added nucleobase at the extended unblocked index primers 414 as A.

When the combined signal is mapped to bin 642 for the base calling cycle, the processor base calls the added nucleobase at the extended read primers 429 as A and the added nucleobase at the extended unblocked index primers 414 as C. When the combined signal is mapped to bin 644 for the base calling cycle, the processor base calls the added nucleobase at the extended read primers 429 as A and the added nucleobase at the extended unblocked index primers 414 as T. When the combined signal is mapped to bin 646 for the base calling cycle, the processor base calls the added nucleobase at the extended read primers 429 as A and the added nucleobase at the extended unblocked index primers 414 as G. When the combined signal is mapped to bin 648 for the base calling cycle, the processor base calls both the added nucleobase at the extended read primers 429 and the added nucleobase at the extended unblocked index primers 414 as A.

### Simplified Sequencing Workflow

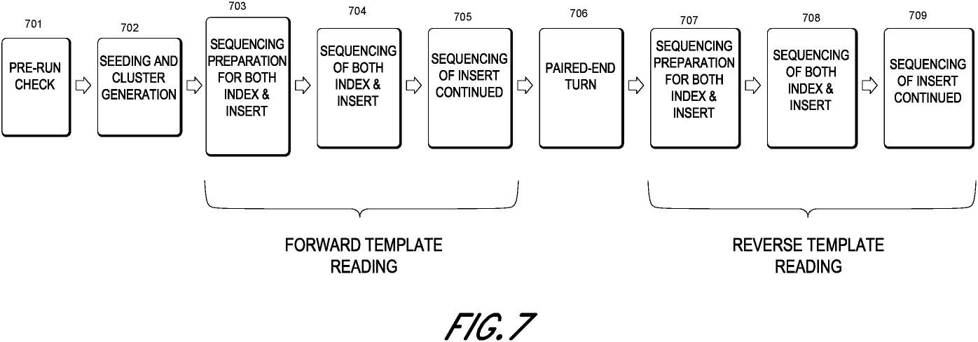

FIG. 7 is a flow diagram showing a simplified method for sequencing a polynucleotide which may utilize the embodiment of the disclosed technology according to FIG. 4. The simplified method may require a shorter sequencing time as index sequencing preparation and insert sequencing preparation (e.g., hybridizing primers) are combined into a single reaction step, for both forward and reverse template sequencings. Moreover, index and insert may be sequenced simultaneously in the first few sequencing cycles (e.g., first 10 cycles), thus saving the time for a few sequencing cycles, for both forward and reverse template sequencings.

As shown in FIG. 7, the disclosed method may start from block 701, a pre-run check of the sequencer and reagent conditions. The method may then move to block 702, seeding and cluster generation, which may include the attachment of a forward template polynucleotide strand to a planar, optically transparent surface on which oligonucleotide anchors are bound. The forward template polynucleotide includes an index portion and an insert portion as explained in FIG. 4. The forward template polynucleotide may be end-repaired to generate 5′-phosphorylated blunt ends, and the polymerase activity of Klenow fragment may be used to add a single A base to the 3′ end of the blunt phosphorylated nucleic acid fragments. This addition prepares the nucleic acid fragments for ligation to oligonucleotide adapters, which have an overhang of a single T base at their 3′ end to increase ligation efficiency. The adapter oligonucleotides are complementary to the flow cell anchor oligos. Under limiting-dilution conditions, adapter-modified, single-stranded forward template polynucleotides are added to the flow cell and immobilized by hybridization to the anchor oligos. Attached nucleic acid fragments are extended and bridge amplified to create an ultrahigh density sequencing flow cell with hundreds of millions of clusters, each containing about 1,000 copies of the same template. Details regarding enrichment of nucleic acids using cluster amplification may be found in Kozarewa et al., Nature Methods 6:291-295 (2009), which is incorporated herein by reference.

After cluster generation, the method may move to block 703, sequencing preparation for both the index and the insert portions of the forward template strand, which includes providing a mixture of primers to the flow cell, and hybridizing/annealing both the index primers and the read primers to the DNA clusters. Next, the method may move to block 704, sequencing of both the index portion and the insert portion of the forward template strand. Sequencing proceeds by extending the primers to generate nucleobase reads. With each cycle, fluorescently tagged nucleotides compete for addition to the growing chains of extended primers. Only one is incorporated at a primer location based on the sequence of the forward template. After the addition of nucleotides, the cluster is excited by a light source, and characteristic fluorescent signals are emitted. The emission spectra and the signal intensities determine the base call. Hundreds of millions of nucleic acid clusters, or thousands to tens of thousands of millions of clusters, may be sequenced in a massively parallel manner. When primer extension of the index primers stops, the method may move to block 705, where sequencing of only the insert portion of the forward template is continued.

After completion of the forward template strand sequencing (i.e., blocks 703-705), the method may move to block 706, paired-end turn. In the paired-end turn process, the primer extension products synthesized during the forward template strand sequencing may be de-hybridized and washed away. Since both the extended read primers and the extended index primers can be de-hybridized and washed away in the same step, the disclosed method can further save sequencing time. Next, the 3′ end of the forward template strand may be deprotected. The forward template strand then folds over and binds to a second oligo on the flow cell. Next, polymerases may be used to extend the second flow cell oligos, forming a double-stranded bridge. This double-stranded nucleic acid bridge may then be denatured, and the 3′ end may be blocked. The original forward template strand may be cleaved off and washed away, leaving the reverse template strand which is complementary to the forward template strand.

After the paired-end turn, the method may proceed with reverse template sequencing. The method may move to block 707, sequencing preparation for both an index portion and an insert portion of the reverse template strand, which includes providing a mixture of (reverse) primers to the flow cell, and hybridizing/annealing both the (reverse) index primers and the (reverse) read primers to the nucleic acid clusters. Next, the method may move to block 708, sequencing of both the index portion and the insert portion of the reverse template strand. Sequencing proceeds by extending the primers to generate nucleobase reads. When primer extension of the (reverse) index primers stops, the method may move to block 709, where sequencing of only the insert portion of the reverse strand is continued.

### Samples

In some embodiments, the sample comprises or consists of a purified or isolated polynucleotide derived from a tissue sample, a biological fluid sample, a cell sample, and the like. Suitable biological fluid samples include, but are not limited to blood, plasma, serum, sweat, tears, sputum, urine, sputum, ear flow, lymph, saliva, cerebrospinal fluid, ravages, bone marrow suspension, vaginal flow, trans-cervical lavage, brain fluid, ascites, milk, secretions of the respiratory, intestinal and genitourinary tracts, amniotic fluid, milk, and leukophoresis samples. In some embodiments, the sample is a sample that is easily obtainable by non-invasive procedures, e.g., blood, plasma, serum, sweat, tears, sputum, urine, sputum, ear flow, saliva or feces. In certain embodiments the sample is a peripheral blood sample, or the plasma and/or serum fractions of a peripheral blood sample. In other embodiments, the biological sample is a swab or smear, a biopsy specimen, or a cell culture. In another embodiment, the sample is a mixture of two or more biological samples, e.g., a biological sample can comprise two or more of a biological fluid sample, a tissue sample, and a cell culture sample. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

In certain embodiments, samples can be obtained from sources, including, but not limited to, samples from different individuals, samples from different developmental stages of the same or different individuals, samples from different diseased individuals (e.g., individuals with cancer or suspected of having a genetic disorder), normal individuals, samples obtained at different stages of a disease in an individual, samples obtained from an individual subjected to different treatments for a disease, samples from individuals subjected to different environmental factors, samples from individuals with predisposition to a pathology, samples individuals with exposure to an infectious disease agent, and the like.

In one illustrative, but non-limiting embodiment, the sample is a maternal sample that is obtained from a pregnant female, for example a pregnant woman. The maternal sample can be a tissue sample, a biological fluid sample, or a cell sample. In another illustrative, but non-limiting embodiment, the maternal sample is a mixture of two or more biological samples, e.g., the biological sample can comprise two or more of a biological fluid sample, a tissue sample, and a cell culture sample.

In certain embodiments samples can also be obtained from in vitro cultured tissues, cells, or other polynucleotide-containing sources. The cultured samples can be taken from sources including, but not limited to, cultures (e.g., tissue or cells) maintained in different media and conditions (e.g., pH, pressure, or temperature), cultures (e.g., tissue or cells) maintained for different periods of length, cultures (e.g., tissue or cells) treated with different factors or reagents (e.g., a drug candidate, or a modulator), or cultures of different types of tissue and/or cells.

In some embodiments, the use of the disclosed sequencing technology does not involve the preparation of sequencing libraries. In other embodiments, the sequencing technology contemplated herein involve the preparation of sequencing libraries. In one illustrative approach, sequencing library preparation involves the production of a random collection of adapter-modified DNA fragments (e.g., polynucleotides) that are ready to be sequenced.

Sequencing libraries of polynucleotides can be prepared from DNA or RNA, including equivalents, analogs of either DNA or cDNA, for example, DNA or cDNA that is complementary or copy DNA produced from an RNA template, by the action of reverse transcriptase. The polynucleotides may originate in double-stranded form (e.g., dsDNA such as genomic DNA fragments, cDNA, PCR amplification products, and the like) or, in certain embodiments, the polynucleotides may originate in single-stranded form (e.g., ssDNA, RNA, etc.) and have been converted to dsDNA form. By way of illustration, in certain embodiments, single stranded mRNA molecules may be copied into double-stranded cDNAs suitable for use in preparing a sequencing library. The precise sequence of the primary polynucleotide molecules is generally not material to the method of library preparation, and may be known or unknown. In one embodiment, the polynucleotide molecules are DNA molecules. More particularly, in certain embodiments, the polynucleotide molecules represent the entire genetic complement of an organism or substantially the entire genetic complement of an organism, and are genomic DNA molecules (e.g., cellular DNA, cell free DNA (cfDNA), etc.), that typically include both intron sequence and exon sequence (coding sequence), as well as noncoding regulatory sequences such as promoter and enhancer sequences. In certain embodiments, the primary polynucleotide molecules comprise human genomic DNA molecules, e.g., cfDNA molecules present in peripheral blood of a pregnant subject.

Methods of isolating nucleic acids from biological sources may differ depending upon the nature of the source. One of skill in the art can readily isolate nucleic acids from a source as needed for the method described herein. In some instances, it can be advantageous to fragment large nucleic acid molecules (e.g. cellular genomic DNA) in the nucleic acid sample to obtain polynucleotides in the desired size range. Fragmentation can be random, or it can be specific, as achieved, for example, using restriction endonuclease digestion. Methods for random fragmentation may include, for example, limited DNase digestion, alkali treatment and physical shearing. Fragmentation can also be achieved by any of a number of methods known to those of skill in the art. For example, fragmentation can be achieved by mechanical means including, but not limited to nebulization, sonication and hydro shear.

In some embodiments, sample nucleic acids are obtained from as cfDNA, which is not subjected to fragmentation. For example, cfDNA, typically exists as fragments of less than about 300 base pairs and consequently, fragmentation is not typically necessary for generating a sequencing library using cfDNA samples.

Typically, whether polynucleotides are forcibly fragmented (e.g., fragmented in vitro), or naturally exist as fragments, they are converted to blunt-ended DNA having 5′-phosphates and 3′-hydroxyl. Standard protocols, e.g., protocols for sequencing using, for example, the Illumina platform, instruct users to end-repair sample DNA, to purify the end-repaired products prior to dA-tailing, and to purify the dA-tailing products prior to the adaptor-ligating steps of the library preparation.

In various embodiments, verification of the integrity of the samples and sample tracking can be accomplished by sequencing mixtures of sample genomic nucleic acids, e.g., cfDNA, and accompanying marker nucleic acids that have been introduced into the samples, e.g., prior to processing.

### Computing Systems

In some embodiments, the disclosed systems and methods may involve approaches for shifting or distributing certain sequence data analysis features and sequence data storage to a cloud computing environment or cloud-based network. User interaction with sequencing data, genome data, or other types of biological data may be mediated via a central hub that stores and controls access to various interactions with the data. In some embodiments, the cloud computing environment may also provide sharing of protocols, analysis methods, libraries, sequence data as well as distributed processing for sequencing, analysis, and reporting. In some embodiments, the cloud computing environment facilitates modification or annotation of sequence data by users. In some embodiments, the systems and methods may be implemented in a computer browser, on-demand or on-line.

In some embodiments, software written to perform the methods as described herein is stored in some form of computer readable medium, such as memory, CD-ROM, DVD-ROM, memory stick, flash drive, hard drive, SSD hard drive, server, mainframe storage system and the like.

In some embodiments, the methods may be written in any of various suitable programming languages, for example compiled languages such as C, C#, C++, Fortran, and Java. Other programming languages could be script languages, such as Perl, MatLab, SAS, SPSS, Python, Ruby, Pascal, Delphi, R and PHP. In some embodiments, the methods are written in C, C#, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly, or are indirectly held on a computer readable medium and loaded as needed onto a computer system. Further, the methods may be located on computers that are remote to where the data is being produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (e.g., iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computers are clustered together to yield a supercomputer network.

In some embodiments, computational methods as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “cluster” or “clump” refers to a group of molecules, e.g., a group of DNA, or a group of signals. In some embodiments, the signals of a cluster are derived from different features. In some embodiments, a signal clump represents a physical region covered by one amplified oligonucleotide. Each signal clump could be ideally observed as several signals. Accordingly, duplicate signals could be detected from the same clump of signals. In some embodiments, a cluster or clump of signals can comprise one or more signals or spots that correspond to a particular feature. When used in connection with microarray devices or other molecular analytical devices, a cluster can comprise one or more signals that together occupy the physical region occupied by an amplified oligonucleotide (or other polynucleotide or polypeptide with a same or similar sequence). For example, where a feature is an amplified oligonucleotide, a cluster can be the physical region covered by one amplified oligonucleotide. In other embodiments, a cluster or clump of signals need not strictly correspond to a feature. For example, spurious noise signals may be included in a signal cluster but not necessarily be within the feature area. For example, a cluster of signals from four cycles of a sequencing reaction could comprise at least four signals.

As used herein, the term “spot radius” or “cluster radius” refers to a defined radius which encompasses a diffraction-limited spot or a cluster of signals. Accordingly, by defining a cluster radius as larger or smaller, a greater number of signals can fall within the radius for subsequent ordering and selection. A cluster radius can be defined by any distance measure, such as pixels, meters, millimeters, or any other useful measure of distance.

As used herein, a “signal” refers to a detectable event such as an emission, such as light emission, for example, in an image. Thus, in some embodiments, a signal can represent any detectable light emission that is captured in an image (i.e., a “spot”). Thus, as used herein, “signal” can refer to an actual emission from a feature of the specimen, or can refer to a spurious emission that does not correlate to an actual feature. Thus, a signal could arise from noise and could be later discarded as not representative of an actual feature of a specimen.

As used herein, an “intensity” of an emitted light refers to the intensity of the light transferred per unit area, where the area is measured on the plane perpendicular to the direction of propagation of the light ray, and where the intensity is the amount of energy transferred per unit time. In some embodiments, signal “strength”, “amplitude”, “magnitude” or “level” may be used synonymously with signal intensity. In some embodiments, an image taken by a detector is approximately or proportional to an intensity map integrated over some amount of time. In some embodiments, the signal of a diffraction-limited spot of a DNA cluster is extracted from the image as the total intensity included in the spot, up to a factor of the integration time. For example, the signal of a DNA cluster may be defined as the intensity included within the spot radius of the DNA cluster, up to a factor of the integration time. In other embodiments, the peak intensity value found within the spot radius may be used to represent the signal of the DNA cluster, up to a factor of the integration time.

As used herein, the process of aligning the template of signal positions onto a given image is referred to as “registration”, and the process for determining an intensity value or an amplitude value for each signal in the template for a given image is referred to as “intensity extraction”. For registration, the methods and systems provided herein may take advantage of the random nature of signal clump positions by using image correlation to align the template to the image.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides may be natural nucleotides, but it is to be further understood that non-natural nucleotides, modified nucleotides or analogs of the aforementioned nucleotides can also be used.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6- diaminopurine, N6-ethano-2,6-diaminopurine, 5-methylcytosine, 5-(C3-C6)- alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

The polymerase used is an enzyme generally for joining 3′-OH 5′-triphosphate nucleotides, oligomers, and their analogs. Polymerases include, but are not limited to, DNA-dependent DNA polymerases, DNA-dependent RNA polymerases, RNA- dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase I, Klenow fragment, Thermophilus aquaticus DNA polymerase, Tth DNA polymerase, VentR@ DNA polymerase (New England Biolabs), Deep VentR® DNA polymerase (New England Biolabs), Bst DNA Polymerase Large Fragment, Stoeffel Fragment, 90N DNA Polymerase, 90N DNA polymerase, Pfu DNA Polymerase, TfI DNA Polymerase, Tth DNA Polymerase, RepliPHI Phi29 Polymerase, TIi DNA polymerase, eukaryotic DNA polymerase beta, telomerase, Therminator® polymerase (New England Biolabs), KOD HiFi® DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting, and polymerases cited in US 2007/0048748, US 6,329,178, US 6,602,695, and US 6,395,524 (incorporated by reference). These polymerases include wild-type, mutant isoforms, and genetically engineered variants. “Encode” or “parse” are verbs referring to transferring from one format to another, and refers to transferring the genetic information of target template base sequence into an arrangement of reporters.

Nucleosides and nucleotides may be labeled at sites on the sugar or nucleobase. A dye may be attached to any position on the nucleotide base, for example, through a linker. In particular embodiments, Watson-Crick base pairing can still be carried out for the resulting analog. Particular nucleobase labeling sites include the C5 position of a pyrimidine base or the C7 position of a 7-deaza purine base. A linker group may be used to covalently attach a dye to the nucleoside or nucleotide. As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

Various different types of linkers having different lengths and chemical properties can be used. The term “linker” encompasses any moiety that is useful to connect one or more molecules or compounds to each other, to other components of a reaction mixture, and/or to a reaction site. For example, a linker can attach a reporter molecule or “label” (e.g., a fluorescent dye) to a reaction component. In certain embodiments, the linker is a member selected from substituted or unsubstituted alkyl (e.g., a 2-5 carbon chain), substituted or unsubstituted heteroalkyl, substituted or unsubstituted aryl, substituted or unsubstituted heteroaryl, substituted or unsubstituted cycloalkyl, and substituted or unsubstituted heterocycloalkyl. In one example, the linker moiety is selected from straight- and branched carbon-chains, optionally including at least one heteroatom (e.g., at least one functional group, such as ether, thioether, amide, sulfonamide, carbonate, carbamate, urea and thiourea), and optionally including at least one aromatic, heteroaromatic or non-aromatic ring structure (e.g., cycloalkyl, phenyl). In certain embodiments, molecules that have trifunctional linkage capability are used, including, but are not limited to, cynuric chloride, mealamine, diaminopropanoic acid, aspartic acid, cysteine, glutamic acid, pyroglutamic acid, S-acetylmercaptosuccinic anhydride, carbobenzoxylysine, histine, lysine, serine, homoserine, tyrosine, piperidinyl-1,1-amino carboxylic acid, diaminobenzoic acid, etc. In certain specific embodiments, a hydrophilic PEG (polyethylene glycol) linker is used.

In certain embodiments, linkers are derived from molecules which comprise at least two reactive functional groups (e.g., one on each terminus), and these reactive functional groups can react with complementary reactive functional groups on the various reaction components or used to immobilize one or more reaction components at the reaction site. “Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like.

Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

In some embodiments, one or more dye or label molecules may attach to the nucleotide base by non-covalent interactions, or by a combination of covalent and non-covalent interactions via a plurality of intermediating molecules. In one example, a nucleotide or a nucleotide analog, being newly incorporated by the polymerase synthesizing from a target polynucleotide, is initially unlabeled. Then, one or more fluorescent labels may be introduced to the nucleotide or nucleotide analog by binding to labeled affinity reagents containing one or more fluorescent dyes. Uses of unlabeled nucleotides and affinity reagents in sequencing by synthesis have been disclosed in U.S. Publication No. 2013/0079232, which is incorporated herein by reference. For example, one, two, three or each of the four different types of nucleotides (e.g., dATP, dCTP, dGTP and dTTP or dUTP) in the reaction mix may be initially unlabeled. Each of the four types of nucleotides (e.g., dNTPs) may have a 3′ hydroxy blocking group to ensure that only a single base can be added by a polymerase to the 3′ end of a copy polynucleotide being synthesized from the target polynucleotide. After incorporation of an unlabeled nucleotide, an affinity reagent may be then introduced that specifically binds to the incorporated dNTP to provide a labeled extension product comprising the incorporated dNTP. The affinity reagent may be designed to specifically bind to the incorporated dNTP via antibody-antigen interaction or ligand-receptor interaction, for example. The dNTP may be modified to include a specific antigen, which will pair with a specific antibody included in the corresponding affinity reagent. Thus, one, two, three or each of the four different types of nucleotides may be specifically labeled via their corresponding affinity reagents. In some embodiments, the affinity reagents may include small molecules or protein tags that may bind to a hapten moiety of the nucleotide (such as streptavidin-biotin, anti-DIG and DIG, anti-DNP and DNP), antibody (including but not limited to binding fragments of antibodies, single chain antibodies, bispecific antibodies, and the like), aptamers, knottins, affimers, or any other known agent that binds an incorporated nucleotide with a suitable specificity and affinity. In some embodiments, the hapten moiety of the unlabeled nucleotide may be attached to the nucleobase through a cleavable linker, which may be cleaved under the same reaction condition as that for removing the 3′ blocking group. In some embodiments, one affinity reagent may be labeled with multiple copies of the same fluorescent dye, for example, 1, 2, 3, 4, 5, 6, 8, 10, 12, 15 copies of the same dye. In some embodiments, each affinity reagent may be labeled with a different number of copies of the same fluorescent dye. In some embodiments, a first affinity reagent may be labeled with a first number of a first fluorescent dye, a second affinity reagent may be labeled with a second number of a second fluorescent dye, a third affinity reagent may be labeled with a third number of a third fluorescent dye, and a fourth affinity reagent may be labeled with a fourth number of a fourth fluorescent dye. In some embodiments, each affinity reagent may be labeled with a distinct combination of one of more types of dye, where each type of dye has a certain copy number. In some embodiments, different affinity reagents may be labeled with different dyes that can be excited by the same light source, but each dye will have a distinguishable fluorescent intensity or a distinguishable emission spectrum. In some embodiments, different affinity reagents may be labeled with the same dye in different molar ratios to create measurable differences in their fluorescent intensities.

A nucleotide analog may be attached to or associated with one or more photo-detectable labels to provide a detectable signal. In some embodiments, a photo-detectable label may be a fluorescent compound, such as a small molecule fluorescent label. Fluorescent molecules (fluorophores) suitable as a fluorescent label include, but are not limited to: 1,5 IAEDANS; 1,8-ANS; 4-methylumbelliferone; 5-carboxy-2,7-dichlorofluorescein; 5-carboxyfluorescein (5-FAM); fluorescein amidite (FAM); 5-carboxynapthofluorescein; tetrachloro-6-carboxyfluorescein (TET); hexachloro-6-carboxyfluorescein (HEX); 2,7-dimethoxy-4,5-dichloro-6-carboxyfluorescein (JOE); VIC®; NED®; tetramethylrhodamine (TMR); 5-carboxytetramethylrhodamine (5-TAMRA); 5-HAT (Hydroxy Tryptamine); 5-hydroxy tryptamine (HAT); 5-ROX (carboxy-X-rhodamine); 6-carboxyrhodamine 6G; 6-JOE; Light Cycler® red 610; Light Cycler® red 640; Light Cycler® red 670; Light Cycler® red 705; 7-amino-4-methylcoumarin; 7-aminoactinomycin D (7-AAD); 7-hydroxy-4-methylcoumarin; 9-amino-6-chloro-2-methoxyacridine; 6-methoxy-N-(4-aminoalkyl)quinolinium bromide hydrochloride (ABQ); Acid Fuchsin; ACMA (9-amino-6-chloro-2-methoxyacridine); Acridine Orange; Acridine Red; Acridine Yellow; Acriflavin; Acriflavin Feulgen SITSA; AFPs-AutoFluorescent Protein-(Quantum Biotechnologies); Texas Red; Texas Red-X conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamine-lsoThioCyanate); True Blue; TruRed; Ultralite; Uranine B; Uvitex SFC; WW 781; X-Rhodamine; X-Rhodamine-5-(and-6)-Isothiocyanate (5(6)-XRITC); Xylene Orange; Y66F; Y66H; Y66W; YO-PRO-1; YO-PRO-3; YOYO-1; interchelating dyes such as YOYO-3, Sybr Green, Thiazole orange; members of the Alexa Fluor® dye series (from Molecular Probes/Invitrogen) which cover a broad spectrum and match the principal output wavelengths of common excitation sources such as Alexa Fluor 350, Alexa Fluor 405, 430, 488, 500, 514, 532, 546, 555, 568, 594, 610, 633, 635, 647, 660, 680, 700, and 750; members of the Cy Dye fluorophore series (GE Healthcare), also covering a wide spectrum such as Cy3, Cy3B, Cy3.5, Cy5, Cy5.5, Cy7; members of the OysterⓇ dye fluorophores (Denovo Biolabels) such as Oyster-500, -550, -556, 645, 650, 656; members of the DY-Labels series (Dyomics), for example, with maxima of absorption that range from 418 nm (DY-415) to 844 nm (DY-831) such as DY-415, -495, -505, -547, -548, -549, -550, -554, -555, -556, -560, -590, -610, -615, -630, -631, -632, -633, -634, -635, -636, -647, -648, -649, -650, -651, -652, -675, -676, -677, -680, -681, -682, -700, -701, -730, -731, -732, -734, -750, -751, -752, -776, -780, -781, -782, -831, -480XL, -481XL, -485XL, -510XL, -520XL, -521XL; members of the ATTO series of fluorescent labels (ATTO-TEC GmbH) such as ATTO 390, 425, 465, 488, 495, 520, 532, 550, 565, 590, 594, 610, 611X, 620, 633, 635, 637, 647, 647N, 655, 680, 700, 725, 740; members of the CAL Fluor® series or Quasar® series of dyes (Biosearch Technologies) such as CAL Fluor® Gold 540, CAL Fluor® Orange 560, QuasarⓇ 570, CAL Fluor® Red 590, CAL Fluor® Red 610, CAL Fluor® Red 635, QuasarⓇ 570, and QuasarⓇ 670. In some embodiments, a first photo-detectable label interacts with a second photo-detectable moiety to modify the detectable signal, e.g., via fluorescence resonance energy transfer (“FRET”; also known as Förster resonance energy transfer).

The fluorescent labels utilized by the systems and methods disclosed herein can have different peak absorption wavelengths, for example, ranging from 400 nm to 800 nm. In some embodiments, the peak absorption wavelengths of the fluorescent labels can be, or be about, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 nm, or a number or a range between any two of these values. In some embodiments the peak absorption wavelengths of the fluorescent labels can be at least, or at most, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm.

The fluorescent labels can have different peak emission wavelength, for example, ranging from 400 nm to 800 nm. In some embodiments, the peak emission wavelengths of the fluorescent labels can be, or be about, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 nm, or a number or a range between any two of these values. In some embodiments the peak emission wavelengths of the fluorescent labels can be at least, or at most, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm.

The fluorescent labels can have different Stokes shift, for example, ranging from 10 nm to 200 nm. In some embodiments, the stoke shift can be, or be about, 10, 20, 30, 40, 50, 60, 70, 80, 90, 100, 110, 120, 130, 140, 150, 160, 170, 180, 190, 200 nm, or a number or a range between any two of these values. In some embodiments, the stoke shift can be at least, or at most, 10, 20, 30, 40, 50, 60, 70, 80, 90, 100, 110, 120, 130, 140, 150, 160, 170, 180, 190, or 200 nm.

In some embodiments, the distance between the peak emission wavelengths of any two fluorescent labels can vary, for example, ranging from 10 nm to 200 nm. In some embodiments, the distance between the peak emission wavelengths of any two fluorescent labels can be, or be about, 10, 20, 30, 40, 50, 60, 70, 80, 90, 100, 110, 120, 130, 140, 150, 160, 170, 180, 190, 200 nm, or a number or a range between any two of these values. In some embodiments, the distance between the peak emission wavelengths of any two fluorescent labels can be at least, or at most, 10, 20, 30, 40, 50, 60, 70, 80, 90, 100, 110, 120, 130, 140, 150, 160, 170, 180, 190, or 200 nm.

A “light source” may be any device capable of emitting energy along the electromagnetic spectrum. A light source may be a source of visible light (VIS), ultraviolet light (UV) and/or infrared light (IR). “Visible light” (VIS) generally refers to the band of electro-magnetic radiation with a wavelength from about 400 nm to about 750 nm. “Ultraviolet (UV) light” generally refers to electromagnetic radiation with a wavelength shorter than that of visible light, or from about 10 nm to about 400 nm range. “Infrared light” or infrared radiation (IR) generally refers to electromagnetic radiation with a wavelength greater than the VIS range, or from about 750 nm to about 50,000 nm. A light source may also provide full spectrum light. Light sources may output light from a selected wavelength or a range of wavelengths. In some embodiments of the invention, the light source may be configured to provide light above or below a predetermined wavelength, or may provide light within a predetermined range. A light source may be used in combination with a filter, to selectively transmit or block light of a selected wavelength from the light source. A light source may be connected to a power source by one or more electrical connectors; an array of light sources may be connected to a power source in series or in parallel. A power source may be a battery, or a vehicle electrical system or a building electrical system. The light source may be connected to a power source via control electronics (control circuit); control electronics may comprise one or more switches. The one or more switches may be automated, or controlled by a sensor, timer or other input, or may be controlled by a user, or a combination thereof. For example, a user may operate a switch to turn on a UV light source; the light source may be applied on a constant basis until it is turned off, or it may be pulsed (repeated on/off cycles) until it is turned off. In some embodiments, the light source may be switched from a continuously-on state to a pulsed state, or vice versa. In some embodiments, the light source may be configured to be brightening or darkening over time.

For operation, the light source may be connected to a power source capable of providing sufficient intensity to illuminate the sample. Control electronics may be used to switch the intensity on or off based on input from a user or some other input, and can also be used to modulate the intensity to a suitable level (e.g. to control brightness of the output light). Control electronics may be configured to turn the light source on and off as desired. Control electronics may include a switch for manual, automatic, or semi-automatic operation of the light sources. The one or more switches may be, for example, a transistor, a relay or an electromechanical switch. In some embodiments, the control circuit may further comprise an AC-DC and/or a DC-DC converter for converting the voltage from the voltage source to an appropriate voltage for the light source. The control circuit may comprise a DC-DC regulator for regulation of the voltage. The control circuit may further comprise a timer and/or other circuitry elements for applying electric voltage to the optical filter for a fixed period of time following the receipt of input. A switch may be activated manually or automatically in response to predetermined conditions, or with a timer. For example, control electronics may process information such as user input, stored instructions, or the like.

One or more of a plurality of light sources may be provided. In some embodiments, each of the plurality of light sources may be the same. Alternatively, one or more of the light sources may vary. The light characteristics of the light emitted by the light sources may be the same or may vary. A plurality of light sources may or may not be independently controllable. One or more characteristic of the light source may or may not be controlled, including but not limited to whether the light source is on or off, brightness of light source, wavelength of light, intensity of light, angle of illumination, position of light source, or any combination thereof.

In some embodiments, light output from a light source may be from about 350 to about 750 nm, or any amount or range therebetween, for example from about 350 nm to about 360, 370, 380, 390, 400, 410, 420, 430 or about 450 nm, or any amount or range therebetween. In other embodiments, light from a light source may be from about 550 to about 700 nm, or any amount or range therebetween, for example from about 550 to about 560, 570, 580, 590, 600, 610, 620, 630, 640, 650, 660, 670, 680, 690 or about 700 nm, or any amount or range therebetween. In some embodiments, the wavelength of the light generated by the light source can vary, for example, ranging from 400 nm to 800 nm. In some embodiments, the wavelength of the light generated by the light source can be, or be about, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 nm, or a number or a range between any two of these values. In some embodiments, the wavelength of the light generated by the light source can be at least, or at most, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm. The light source may be capable of emitting electromagnetic waves in any spectrum. In some embodiments, the light source may have a wavelength falling between 10 nm and 100 µm. In some embodiments, the wavelength of light may fall between 100 nm to 5000 nm, 300 nm to 1000 nm, or 400 nm to 800 nm. In some embodiments, the wavelength of light may be less than, and/or equal to 10 nm, 100 nm, 200 nm, 300 nm, 400 nm, 500 nm, 600 nm, 700 nm, 800 nm, 900 nm, 1000 nm, 1100 nm, 1200 nm, 1300 nm, 1500 nm, 1750 nm, 2000 nm, 2500 nm, 3000 nm, 4000 nm, or 5000 nm.

In one example, a light source may be a light-emitting diode (LED) (e.g., gallium arsenide (GaAs) LED, aluminum gallium arsenide (AlGaAs) LED, gallium arsenide phosphide (GaAsP) LED, aluminum gallium indium phosphide (AlGaInP) LED, gallium(III) phosphide (GaP) LED, indium gallium nitride (InGaN)/gallium(III) nitride (GaN) LED, or aluminum gallium phosphide (AlGaP) LED). In another example, a light source can be a laser, for example a vertical cavity surface emitting laser (VCSEL) or other suitable light emitter such as an Indium-Gallium-Aluminum-Phosphide (InGaAIP) laser, a Gallium-Arsenic Phosphide/Gallium Phosphide (GaAsP/GaP) laser, or a Gallium-Aluminum-Arsenide/Gallium-Aluminum-Arsenide (GaAIAs/GaAs) laser. Other examples of light sources may include but are not limited to electron stimulated light sources (e.g., Cathodoluminescence, Electron Stimulated Luminescence (ESL light bulbs), Cathode ray tube (CRT monitor), Nixie tube), incandescent light sources (e.g., Carbon button lamp, Conventional incandescent light bulbs, Halogen lamps, Globar, Nernst lamp), electroluminescent (EL) light sources (e.g., Light-emitting diodes-Organic light-emitting diodes, Polymer light-emitting diodes, Solid-state lighting, LED lamp, Electroluminescent sheets Electroluminescent wires), gas discharge light sources (e.g., Fluorescent lamps, Inductive lighting, Hollow cathode lamp, Neon and argon lamps, Plasma lamps, Xenon flash lamps), or high-intensity discharge light sources (e.g., Carbon arc lamps, Ceramic discharge metal halide lamps, Hydrargyrum medium-arc iodide lamps, Mercury-vapor lamps, Metal halide lamps, Sodium vapor lamps, Xenon arc lamps). Alternatively, a light source may be a bioluminescent, chemiluminescent, phosphorescent, or fluorescent light source.

As used herein, an “optical channel” is a predefined profile of optical frequencies (or equivalently, wavelengths). For example, a first optical channel may have wavelengths of 500 nm-600 nm. To take an image in the first optical channel, one may use a detector which is only responsive to 500 nm-600 nm light, or use a bandpass filter having a transmission window of 500 nm-600 nm to filter the incoming light onto a detector responsive to 300 nm-800 nm light. A second optical channel may have wavelengths of 300 nm-450 nm and 850 nm-900 nm. To take an image in the second optical channel, one may use a detector responsive to 300 nm-450 nm light and another detector responsive to 850 nm-900 nm light and then combine the detected signals of the two detectors. Alternatively, to take an image in the second optical channel, one may use a bandstop filter which rejects 451 nm-849 nm light in front of a detector responsive to 300 nm-900 nm light.

### Additional Notes

The embodiments described herein are exemplary. Modifications, rearrangements, substitute processes, etc. may be made to these embodiments and still be encompassed within the teachings set forth herein. One or more of the steps, processes, or methods described herein may be carried out by one or more processing and/or digital devices, suitably programmed.

The various illustrative imaging or data processing techniques described in connection with the embodiments disclosed herein can be implemented as electronic hardware, computer software, or combinations of both. To illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative detection systems described in connection with the embodiments disclosed herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory.

The elements of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (e.g., X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (e.g., measurement value) is close to a targeted value, where close can mean, for example, the result is within 80% of the value, within 90% of the value, within 95% of the value, or within 99% of the value.

Unless otherwise explicitly stated, articles such as “a” or “an” should generally be interpreted to include one or more described items. Accordingly, phrases such as “a device configured to” or “a device to” are intended to include one or more recited devices. Such one or more recited devices can also be collectively configured to carry out the stated recitations. For example, “a processor to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

What is claimed is:

1. A method of identifying nucleobases in a template polynucleotide, the method comprising:
providing a substrate comprising a plurality of the template polynucleotides in a cluster;
generating light to stimulate fluorescent emissions from the cluster;
receiving a first signal emitted at a first intensity from a first plurality of nucleotide analogs hybridized to the plurality of template polynucleotides at a first site;
receiving a second signal emitted at a second intensity from a second plurality of nucleotide analogs hybridized to the plurality of template polynucleotides at a second site; and
identifying the nucleobases hybridized at the first and second sites of the template polynucleotide based on a combination of the first and second signals.

2. The method of claim 1, wherein the first site is in a barcode index segment of the template polynucleotide, and wherein the second site is in a non-barcode index segment of the template polynucleotide.

3. The method of claim 1, comprising identifying the nucleobases in the template polynucleotide based on the difference between the first intensity and the second intensity of the first and second signals.

4. The method of claim 1, wherein the nucleotide analogs in the first plurality of nucleotide analogs are part of index primers hybridized to a site adjacent to a barcode index portion of the template polynucleotides in the cluster.

5. The method of claim 4, wherein the nucleotide analogs in the second plurality of nucleotide analogs are part of read primers hybridized to a site adjacent to a non-barcode index portion of the template polynucleotides in the cluster.

6. The method of claim 5, wherein identifying the nucleobases comprises comparing the first signal at the first intensity from the nucleotide analogs on the index primers with the second signal at the second intensity from nucleotide analogs on the read primers.

7. The method of claim 6, wherein the first signal has an intensity that is lower than the intensity of the second signal.

8. The method of claim 5, wherein the intensity of the first and second signals corresponds with the concentrations of the first plurality of nucleotide analogs in the index primers and the second plurality of nucleotide analogs in the read primers.

9. The method of claim 5, wherein a fraction of the index primers have a blocked 3′end, and the read primers have an unblocked 3′-end.

10. The method of claim 1, further comprising substantially simultaneously identifying nucleobases in an additional polynucleotide complementary to the template polynucleotide.

11. A method of determining the sequence of a template polynucleotide comprising a barcode index, comprising:
hybridizing an index primer and a read primer to the template polynucleotide;
extending the index primer with a first labeled nucleotide comprising a first label;
extending the read primer with a second labeled nucleotide comprising a second label;
generating light to stimulate fluorescent emissions from the first and second labeled nucleotides; and
determining the sequence of nucleotides in the barcode index and the template polynucleotide by capturing the fluorescent emissions.

12. The method of claim 11, wherein the template polynucleotide is part of a cluster of identical copies of the template polynucleotide, wherein the index primer is part of a first population of index primers, and wherein the read primer is part of a second population of read primers.

13. The method of claim 12, wherein determining the sequence of nucleotides in the barcode index and the template polynucleotide comprises:
receiving a first signal emitted at a first intensity from the first population of index primers;
receiving a second signal emitted at a second intensity from the second population of read primers; and
identifying a nucleobase hybridized to the barcode index and a nucleobase hybridized to the template polynucleotide based on a combination of the first and second signals.

14. The method of claim 12, wherein a portion of the first population of index primers have a blocked 3′-end.

15. The method of claim 12, wherein the second population of read primers have an unblocked 3′-end.

16. The method of claim 11, wherein the first labeled nucleotide and the second labeled nucleotide are hybridized to the template polynucleotide in the same reaction step.

17. The method of claim 11, wherein extending the index primer is catalyzed by a non-strand displacing polymerase.

18. The method of claim 11, wherein the index primer is thermally de-hybridized after a predetermined number of nucleic acid polymerization cycles.

19. The method of claim 11, wherein the index primer is removed after a predetermined number of nucleic acid polymerization cycles by contacting a locked nucleic acid with the template polynucleotide.

20. The method of claim 17, comprising further extending the read primer and determining the sequence of nucleotides in the template polynucleotide after extension of the index primer has seized.

21. The method of claim 12, comprising adjusting the ratio of the first population of index primers and the second population of read primers prior to extending the index primer and the read primer.

22. The method of claim 21, comprising:
monitoring the ratio of the first population of index primers and the second population of read primers hybridized to the cluster of identical copies of the template polynucleotide, by stimulating and detecting fluorescent emissions from fluorophores attached to the first population of index primers and the second population of read primers; and
removing the first population of index primers and the second population of read primers at different rates by regulated thermal de-hybridization until a predetermined ratio is achieved.

23. The method of claim 22, further comprising removing the fluorophores attached to the first population of index primers and the second population of read primers remaining hybridized to the cluster.

24. A system of identifying nucleobases in a template polynucleotide, comprising:
a substrate comprising a plurality of the template polynucleotides in a cluster;
a processor configured to perform a method comprising:
generating light to stimulate fluorescent emissions from the cluster;
receiving a first signal emitted at a first intensity from a first plurality of nucleotide analogs hybridized to the plurality of template polynucleotides at a first site;
receiving a second signal emitted at a second intensity from a second plurality of nucleotide analogs hybridized to the plurality of template polynucleotides at a second site; and
identifying the nucleobases hybridized at the first and second sites of the template polynucleotide based on a combination of the first and second signals.

25. The system of claim 24, wherein the processor is configured to identify the nucleobases in the template polynucleotide based on the difference between the first intensity and the second intensity of the first and second signals.
